(19) United States
(12) Patent Application Publication
(10) Pub. No.: US 2011/0307039 A1
(43) Pub. Date: Dec. 15, 2011

(54) Device, method and system for treatment of sinusitis

(76) Inventor: Thomas H. Cornell, Philadelphia, PA (US)

(21) Appl. No.: 12/802,781

(22) Filed: Jun. 14, 2010

(51) Int. Cl. A61F 7/12

(52) U.S. Cl. 607/105

(57) ABSTRACT

A device, method and system for facilitating treatment of a sinusitis condition, including a heat source configured to deliver an output of heated air to the nasal passages of an individual, and a delivery component having a space through which the heated air output of the heat source may pass, the delivery component being configured for placement in the individual's nostrils. The system provides a heated air output source and one or more delivery components configured and sized to fit within the nostril of an individual, which may be used to deliver one or more courses of heated air to a user's sinuses through a nostril.

### BACKGROUND OF THE INVENTION

1. Field of the Invention

The invention relates to devices, methods and systems, including kits, for facilitating the treatment of conditions tending to affect the sinuses, in particular, conditions known as sinusitis.

2. Brief Description of the Related Art

The paranasal cavities are generally located in the head and cheekbones, and are mucus-lined cavities that are air-filled. These paranasal cavities drain into the nasal cavity. Just under the eye sockets are the maxillary sinuses, which are the largest. Frontal sinuses are located in the frontal bone above and between the eye sockets. Other sinuses include the sphenoid and ethmoid sinuses which are located deeper in the skull in the location above the nasal cavity. The nasal cavity is lined with tiny hair-like particles known as cilia. When an individual suffers from the condition known as sinusitis, the mucosal membranes of the sinuses become inflamed. It is not uncommon for colds to progress to the level of sinusitis. If an infection occurs, the lining of the sinuses generally swells, and the swelling, in turn, causes retention of the secretions. This leads to potential bacterial growth in the mucus. Sinusitis generally causes the individual to experience pain in the sinus area (and tangential areas) and may extend to areas such as an individual's teeth. Post-nasal drip becomes so relentless that coughing and noisy throat clearing becomes chronic, causing public embarrassment and sleepless nights.

In many cases, the sinusitis must be treated with antibiotics. Other treatments that may be given include antihistamines, decongestants and pain relievers. The sinusitis condition, however, even with treatment, may last for a considerable time, such as about two weeks. And in many cases, after antibiotic seems to have resolved the condition, the symptoms slowly return. Those are the individuals who experience a chronic sinusitis condition which may go untreated, or may be unresponsive to treatment attempts. These individuals may undergo surgery to remedy their sinusitis condition, but only the most extreme cases are considered medically eligible.

Generally, individuals suffering from sinusitis may have lifelong problems with the condition and suffer from frequent reoccurrences. Physiology of an individual may affect the likelihood of being affected by the condition. For example, individuals with narrow sinuses or septum conditions may be susceptible to sinus problems. For these individuals, colds may run their course, but, inevitably, may lead to prolonged sinusitis symptoms that may eventually progress into major infections. Some individuals have been known to suffer for many weeks with a low-level sinus infection, and, in some instances, doctors are reluctant to give antibiotics unless a patient also has a fever. When an individual suffers from such chronic sinusitis, the individual may experience month after month of compulsive coughing, and, in bed at night, may suffer relentless post-nasal drip causing sleep deprivation.

Patients who complain of these sinusitis symptoms are often thought to be, and indeed often are, sinus allergy sufferers as well, and are therefore prescribed such medications as ceterizine hydrochloride (sold under the brand name Zyrtec®) or the various nasal spray inhalers that may include mometasone furoate (sold under the brand name Nasonex®). Other types of treatment frequently recommended for these symptoms involve saline solution sprays, “neti pot” saline solution irrigation methods, and the use of sinus irrigation water-pik devices that usually include the diffusion of chemicals that can cause unwelcome side effects, as can the many decongestant sprays, gels, and tablets on the market, whether prescribed or over the counter. For example, prolonged reliance on a product containing Sudafed® (or pseudoephedrine) by a desperate sinusitis sufferer may result in renal failure. While any of these treatments may provide temporary relief of symptoms, they can be messy to administer, risky to rely on, and provide no long-term confidence that public coughing attacks and sleepless nights of struggling with post-nasal drip will come to an end.

The market is filled with home remedy treatment methods and systems attempting to address this medical problem that is said to afflict more than 40% of the population, and the vast majority of these are based on the use of water, whether in liquid form or that of vapor, with various admixtures of salt or medications in each. The bias for water-based approaches is because it has been generally accepted that humidity is the great sinus healer. This assumption is based on the proposition that the nose is supposed to moisten inhaled air, to maintain a moist environment for the healthy functioning of the hair-like cilia inside the nasal cavities. This is why everything water-based, from a water pik to the “steam tent,” in which one's face is held over a pot of boiling water with a towel over the head to trap the inhaled vapor, has been the home remedy treatment approach of choice. Examples include a product identified as the “Breathe-Ease Nasal Spray and Irrigation Solution,” advertised to extol the importance of providing the sinus with artificial saline moisturizing, although there is also mention of the potential for some other nasal spray products which include preservative additives to actually irritate the sinus. Another available treatment is through a company named Sinus Dynamics wherein a subject inhales alternately through each nostril from the narrow end, inserted into the nostril, of a bulb connected to a nebulizing device that delivers aerosolized medication into the sinus. Because of how negative ions are reputed to have respiratory cleansing properties, another home remedy treatment involving the inhalation of crystal-salt-infused air has become popular. A product referred to as the Himalayan Crystal Salt inhaler, while making no claims regarding sinusitis, does claim general respiratory system benefits to inhaling crystal salt ions through the mouth and exhaling through the nose.

According to Dr. Terence Davidson at the University of California, San Diego School of Medicine, the sinus tissue is called mucosa. There are little hairs called cilia and a layer of fluid floating over the cilia. The cilia propel a blanket of mucus out along this underlying fluid to the back of the throat, where about one quart of mucus is swallowed each day. Dust and bacteria is trapped in the mucus blanket, and this “mucociliary transport system” is necessary to dispose of this unwelcome waste into the stomach and to be later excreted. In the healthy system, this blanket of mucus traveling from sinus to the back of the throat is not noticed. But in an impaired system, nasal and sinus secretions stagnate, and an otherwise automatic process becomes a conscious preoccupation.

The mucociliary transport system becomes paralyzed when the drainage hole between the paranasal passages and the nasal passages, called the “ostium,” becomes swollen shut. The bacteria that is present in the mucus begins to thrive and grow when the mucus blanket has stopped moving, causing infection of the mucosa.

The two most common initial causes of mucosa swelling are catching a cold and reactions to allergens. This swelling is presumably enough to start the vicious circle of stagnation and bacterial breeding that can result in first acute, and later chronic, sinusitis.

Other causes of mucosal inflammation include fungi (such as aspergillus) and vasomotor rhinitis, which according to an online article called “We Live In Interesting Times” (liveonearth.livejournal.com/209975.html) includes humidity in a list of environmental sensitivities that can contribute to a sinusitis condition. There is a growing belief that the substituting of artificial fluids in sprays and fluid irrigation of the sinus, while having some cleansing value, may perpetuate a counter-productive dependence. C. Opitz, an inventor whose patent disclosures will be cited later in this disclosure, has written: “Inhalation of steam has the added disadvantage that it moistens the epithelium externally and thus prompts the body to reduce its own mucus production that serves as part of its defense.”

A proposed aid for sinusitis sufferers is a sinus irrigator Water Pik®. This device provides for a stream of pressurized water directed into the sinus through each nostril alternately. A sinus irrigator tip for the Water Pik® also has been proposed. It is claimed that this pulsate irrigation not only washes out the sinus and replaces bacteria laden mucus with a layer of thin saline fluid to help the cilia function, but the pulsation “moves the cilia back and forth, which helps restore the movement of the cilia.” Improving ciliary function through the intrusion within the sinus passages of pulsate action will prove to be an important feature of this disclosure.

According to an article on WebMD (an online Web site) offering a proposed treatment overview of sinusitis, it is stated that “[s]inusitis is treated with medications and home treatment methods such as applying moist heat to your face.” (webmd.com/a-to-z-guides/sinusitis-treatment-overview) Another on-line article, at herbldoc.com/Sinus.htm, endorses the steam bath treatment, and then includes the statement: “[No]t towels applied over the face can also be helpful . . . .”

U.S. Pat. No. 7,100,605 B2 issued to C. Opitz states that “certain so-called face saunas, for the isolated treatment of the head region, were developed for treating colds. Also, for a long time now, at least in Europe, dry saunas have been recommended by so-called holistic therapists.” Opitz explains that this treatment of the common cold through inhaling dry heat is based on virological research that has established since 1971 that the rhinovirus, known to cause common colds, is not capable of replication at the normal body temperature of 37° C., or 98.6° F. Therefore, this virus can only exist within the epithelial layers of the nasal and throat passages where inflowing air when inhaling causes a lower temperature than the central body. When a study carried out in 1980 at Harbord Hospital in Salisbury, England, established that other viruses responsible for the common cold, the corona viruses in particular, were also incapable of survival at normal central body temperature, the introduction of sustained heat to the nasal and throat passages was, for many, conclusive as the cure for the common cold.

An important influence on Opitz, cited in his patent, is another inventor named R. S. Krauser (see U.S. Pat. No. 5,038,769), whose invention for the treatment of the common cold also relies on the principle of what he terms “hyperthermia.” He reports that temperatures of higher levels, as high as 150° F., were shown to be more effective in treating virus infections of the sinus and throat than, say, 106 degrees F. He insists that beyond the killing of viruses, controlled heat “also increases the circulation of the blood to the affected area, aiding in the carrying away of dead cell material as well as the rebuilding of new cells.” Krauser goes further in his estimation of the healing potential of heat therapy: “Medical researchers around the world report that heating malignant tissue to the hyperthermic range appears to be a safe and efficient means of either destroying the cancer cells or making them more susceptible to other forms of treatment.”

There are other examples of the healing merits of hyperthermia. For example, cutaneous (skin) therapy has been mentioned in a 2005 study by the Royal Society of Tropical Medicine and Hygiene, authored by Iza M. F. Loba, et al., entitled “Heat therapy for cutaneous leishmaniasis elicits a system cytokine response similar to that of antimonial (Glucantime) therapy,” published by Elsevier Ltd. This article refers to the treatment of skin lesions, infections caused by protozoal parasites, using a ThermSurgery instrument, powered by batteries, which is placed at the edge of the lesion with applied heat of about 50 degrees C. A dramatic healing response was noted to have occurred. Another example of heat therapy is disclosed in U.S. Pat. No. 5,534,021 by Israel Dvoretsky et al., for an invention that involves heating pads and a machine that delivers constant heat to the skin or body. The patent disclosure claims that the sustained application of heat is an effective treatment for a variety of skin maladies, including the treatment of warts.

The patent disclosures of Opitz and Krauser both apply the principle of heat therapy to the treatment of the nasal passages and throat, although only in connection with treating the common cold. Krauser's invention extends its claims to include the treating of various skin maladies. Both use the basic model of a common hair dryer to describe the heating source and forced hot air mechanics of each different device. Krauser's device involves the admixture of a povidone-iodine agent, or alternatively Hexylresorcinal, installed within the body of the device and dispersed with the emission of hot air from the device. Opitz's device also involves an admixture of something housed within it, but in this case it is crystallized salt emitting negative ions as the heated air blows across the salts within the device. In both cases, the means of delivering heated air into the sinus passages requires inhalation of the heated air by the subject either through a face mask or simply by the proximity of the subject's two nostrils to the delivery end of the device. Neither of these devices provides for the sustained delivery of heated air deep within the sinus cavities needed for the treatment of sinusitis, a delivery of heated air with sufficient force to massage and enliven damaged cilia and stimulate the natural secretion of sub-mucosal fluid needed by the cilia.

A need exists for a system, method and device that may be useful for treatment of sinusitis conditions, that may be safe and easy to use and economical to produce, and that provides advantages over prior treatment methods.

### SUMMARY OF THE INVENTION

A device, system, and method for preventing or facilitating the treatment of sinusitis conditions and/or symptoms is provided. The method involves the application of heated air delivery through the nostrils and into the sinuses of an individual. Preferably, the air velocity is regulated and the temperature of the heated air is regulated for the effect and/or comfort of the individual.

The device, system and method preferably facilitate stimulation of mucociliary motion with the application of forced air and heat, which preferably, are delivered together.

According to one embodiment, a device includes a heated air delivery source and a delivery component that directs a heated air stream into the preferred treatment area, which are the sinuses of the individual. The air velocity and heat may be regulatable so that the individual may select combinations of air temperature and air speed. Preferably, the device is configured so that the user may hold the device and have access to these controls.

According to preferred embodiments, the controls may be positioned proximate to a handle, so the user may hold the device and control the settings (e.g., air temperature and air velocity) with the same hand.

Another preferred embodiment may also incorporate the feature of a HEPA filtering system at the rear of the heated air delivery source where air is drawn into the device. The purpose is to filter out allergens from the air that is ultimately propelled into the sinuses of the subject. Filters may be either reusable after washing, or replaceable, easily installed and removed from the cavity at the rear of the source device where a hinged cylindrical lid with screened panels can be opened and latched shut.

According to another embodiment, a device for preventing sinusitis symptoms or facilitating the treatment of sinusitis conditions and/or symptoms is configured for attachment to an existing hair dryer.

According to a preferred embodiment, the attachment device includes a single spout or nozzle, which may be positioned in a nostril of an individual. This device, preferably, is also constructed from heat-resistant materials or has a cover on the nozzle portion so that the individual using it suffers no discomfort from the heat of the air passing through this delivery component device.

According to preferred embodiments, the spout of the delivery component, in this case a component configured for attachment to a hair dryer, may be universally dimensioned so that it may be used by a variety of individuals.

Optionally, the system may be provided as a kit, containing both its own heated air output source, HEPA filters for the output source device, and one or more delivery components. According to an alternate embodiment, the delivery components may be sized for different-sized adult users. The potential for use with children and infants would be determined by medical specialists. Delivery components may be sized to have different nasal penetration depths. In addition, a kit of delivery components having a plurality of sizes also may be provided, as well as a variety of colors so that different family members, for example, could identify and maintain his or her own device while sharing the same hot air delivery system or the same dishwasher in which the different devices are washed.

In addition, kits may or may not be supplied with a heating source device and may include one or more delivery components of the same or different sizes, or shape designs, or special engineering with respect to configuring them for attachment to different-sized hair drying nozzles. For one preferred embodiment of the delivery component's design, such a kit may include one universal delivery component together with a variety of size adaptor rings designed to accommodate different-sized hair dryer nozzles and still be universally attachable to the one delivery component.

The device, system, and method described and illustrated herein may be useful as an alternative to prior treatments for sinusitis, and alternately, or optionally, may be used with one or more prior treatments, such as, for example, antibiotics or saline irrigation. However, the treatment of the present invention has been shown to be useful itself, without other treatments.

The method preferably involves administration of one or more treatment sessions in which heated air is forced into the nasal passages, preferably for a few minutes at a time during each session.

According to preferred embodiments, multiple sessions of heated air delivery to the nasal passages, for a few minutes at a time, with a few minutes between each session may be carried out.

According to a preferred method, treatment by heated air delivery to the sinuses through the nostrils of a person may be done in the morning and in the evening.

These and other advantages of the invention are indicated by the description that follows and the appended claims, and the invention is to be broadly construed in connection with the preferred embodiments disclosed herein.

### BRIEF DESCRIPTION OF THE DRAWING FIGURES

FIG. 1 is a front elevation view of an exemplary embodiment of a first embodiment of a sinusitis treatment device constructed in accordance with my invention shown in an environment with a conventional hair dryer.

FIG. 2 is a perspective view of the device of FIG. 1 looking at the device from the bottom.

FIG. 3 is a front elevation view of a first alternate embodiment of a sinusitis treatment device constructed according to my invention.

FIG. 4 is a perspective view of the device of FIG. 3 looking at the device from the bottom.

FIG. 5 is a perspective view of the device shown in FIGS. 3-4, illustrated in use with a conventional hair dryer.

FIG. 6 is a front elevation view of a second alternate embodiment of a sinusitis treatment device constructed according to my invention, shown in partial sectional view.

FIG. 7 is a perspective view of a third alternate embodiment of a sinusitis device according to my invention as viewed at an angle looking at the bottom.

FIG. 8 is a bottom plan view of the third alternate embodiment of the device shown in FIG. 7.

FIG. 9 is a side elevation view of the third alternate embodiment shown in FIGS. 7-8.

FIG. 10 is a front elevation view of the third alternate embodiment shown in FIGS. 7-9.

FIG. 11 is a fourth alternate embodiment of a sinusitis treatment device constructed in accordance with my invention shown in an environment with a conventional hair dryer.

FIG. 12 is a bottom plan view of the fourth alternate embodiment of a sinusitis treatment device constructed according to my invention.

FIG. 13 is a perspective view of the device of FIG. 11-12 looking at the device from the bottom.

FIGS. 14-16 show a fifth alternate embodiment of a sinusitis treatment device, where FIG. 14 is a perspective view looking at the device from the bottom, FIG. 15 is a bottom plan view, and FIG. 16 is a partial sectional view taken through line 16-16 of FIG. 15.

FIG. 14a is another alternate embodiment having a removable end cap.

FIGS. 17-19 show a sixth alternate embodiment of a sinusitis treatment device, where FIG. 17 is a perspective view looking at the device from the bottom, FIG. 18 is a bottom plan view, and FIG. 19 is a partial sectional view taken through line 19-19 of FIG. 18.

### DETAILED DESCRIPTION OF THE PREFERRED EMBODIMENTS

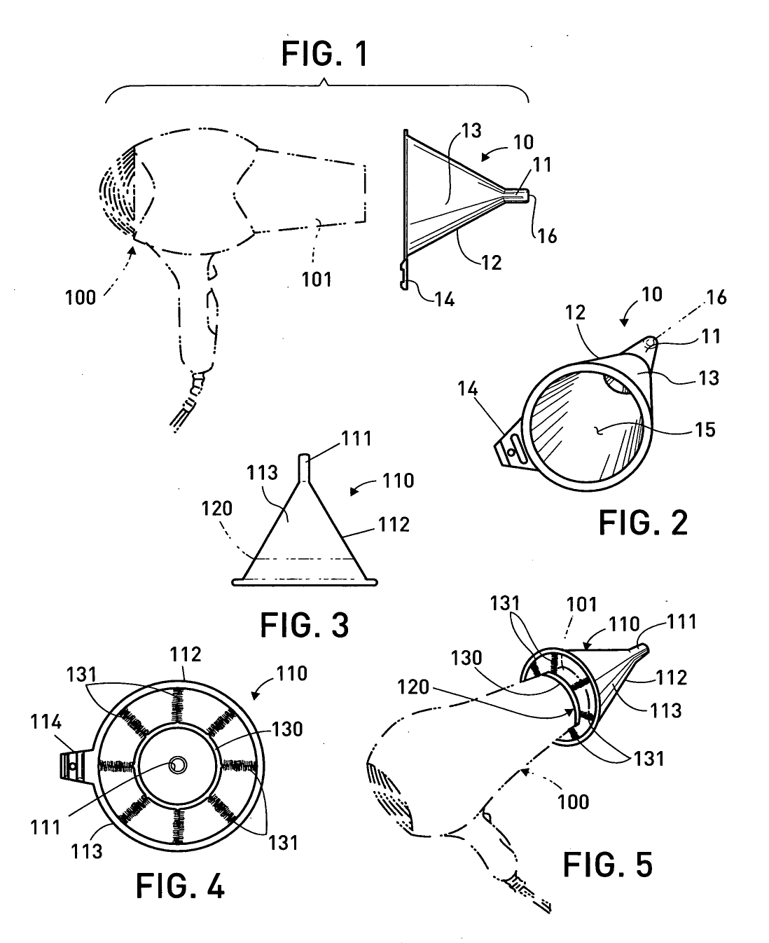

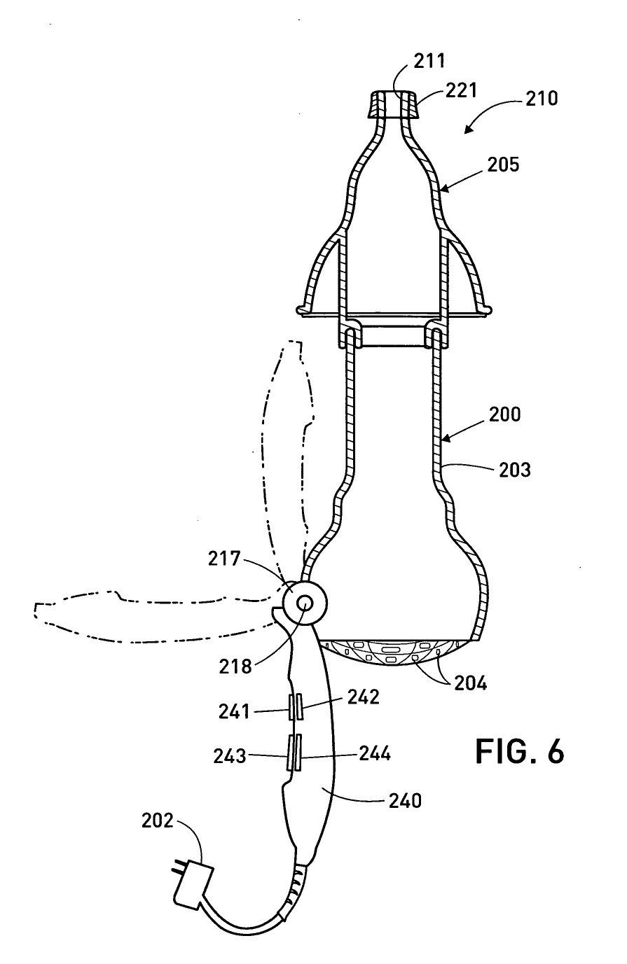

A system, method, and device are provided for treating sinusitis conditions to prevent or alleviate symptoms of sinusitis and sinusitis conditions. Referring to FIG. 1, a preferred embodiment of a sinusitis treatment device 10 is provided configured as a delivery component for delivering a flow of heated air to a person. Preferably, the device 10 includes one or more delivery portions, such as, for example, the nozzle 11. The device 10 has a body 12 which has a wall 13, a handle 14, and a space 15 therein (FIG. 2) so that heated air may be delivered through the spout or nozzle 11 of the device 10. The nozzle 11 has an opening 16 through which heated air may be delivered into a nostril of a user. According to a preferred embodiment illustrated in FIGS. 1 and 2, the device 10 is configured for use with a heated air source, such as, for example, the hand held hair dryer 100. The hair dryer 100 may, for example, be any of those commercially available hair dryers sold for household use (or even those sold for professional use such as in a barber shop or beauty parlor). Preferably, the spout or nozzle 11 is constructed from a material that does not raise its temperature to a degree that will otherwise burn the skin of a human. Alternately, the nozzle 11 may be coated, such as, for example, with a plastic coating or covering, such as a plastic end cap (see e.g., FIGS. 5 and 6), that is resistant to heating to an elevated temperature that would otherwise cause a burn to human skin (when the heated air of a heated air output device is used with the device 10).

Though the device 10 may be used by holding the device 10 and directing a flow of heated air through the device 10, according to an alternate embodiment illustrated in FIGS. 3-5, a sinusitis treatment device 110 is provided, and is configured similar to the device 10 shown in FIGS. 1 and 2, but includes a connector 120 for connecting the device 110 to a heated air source, such as, for example, the hair dryer 100. Preferably, the connector 120 may include an elastomeric band 130 that may be maneuvered to fit on the heat source, such as being stretched to fit over the hair dryer end or nozzle 101 (FIG. 5). The band 130 is attached to the wall 113 of the body 112 of the device 110 with a suitable attachment component, such as, for example, the springs 131. The springs 131 are spaced apart and hold the band 130 on the device 110. The spacing and arrangement of the springs 131 permits ventilation when a heated air source, such as the hair dryer 100, is attached to the band 130 and delivering heated air. Suitable securing elements, such as, for example, a molded holder, clips, rivets, adhesives or the like may be used to secure an end of each spring 131 to the device wall 113. The springs 131 preferably have ends, including one end that may be connected to the band 130 and another end that is connected to the wall 113. According to a preferred embodiment, the springs 131 may extend through the wall 113 of the device 110 and be secured thereto. The band 130 may be continuously provided, or alternately, may be overlapped at its ends, see for example, the ends 130a, 130b of FIG. 4, to provide a suitable range of dimensions.

Other suitable connectors, though not shown, also may be used, such as, for example, lugs, threads, bayonet fittings, or the like. Preferably, the connector 120 and, in the embodiment illustrated, the elastomeric band 130, or other connector utilized, is constructed from a material which does not elevate its temperature (or which does not significantly elevate its temperature, or alternatively, which may rapidly cool, after being exposed to heat. Preferably a heat resistant material is used to construct the connector 120. Preferably, the elastomeric band 130 is configured to make a secure engagement with the dryer 100.

Referring to FIG. 6, according to an alternate embodiment, a device 210 comprising a delivery component 205 and a heated air output unit 200 is shown. The delivery component 205 has a nozzle 211 which may be constructed similar to the nozzles 11 and 111, of the embodiments of the devices 10 and 110, respectively, illustrated in FIGS. 1-5 and described herein. The nozzle 211 optionally may be provided with an end cap 221 to facilitate heat resistance, and prevent potential burning of the user. The optional nozzle cap 221 is shown on the nozzle 211 of the device 210 in FIG. 6. The nozzle cap 221 may be provided for comfort or to minimize heat transfer from the nozzle 211, or both. Where a device or nozzle of the device is constructed from a heat resistant material (e.g., a material which when exposed to the heated air does not elevate its temperature to a level that is uncomfortable or may burn a user), the device may be used without the nozzle cap 221. The nozzle cap 221, though shown on the device 210 illustrated in FIG. 6, optionally may be used in connection with other embodiments shown and described herein.

The delivery component 205 and heated air output unit 200 may be provided as a system in a kit form. Accordingly, the delivery component 205, as with the device 10 and device 110, may be provided, having different sizes to accommodate users having different size nasal openings (e.g., nostrils). Preferably, the nozzle 211, as well as the nozzles 11, 111, are constructed to have a size that may be universally usable by a variety of individuals. Alternately, for example, the nozzle 221 (as well as other nozzles disclosed in other embodiments shown and described herein) may be configured having different lengths and dimensions (e.g., widths or diameters) so that the insertion into the nostrils of a user may be controlled and may be comfortable. For example, the nozzle 211 may be sized smaller, or conversely, the nozzle 211 may be larger. Though the nozzles 11, 111, 211 are shown having a particular configuration, the nozzles may be provided having other shapes, such as, for example, being tapered or oblong.

According to a preferred embodiment, the device 210 may be provided with a handle 240 for facilitating holding the device 210 so a user may apply treatment as desired. Though a single handle 240 is shown, one or more additional handles (not shown) may be provided. Alternately, the handle 240 may be provided within the perimeter of the device 210 to provide a more compact configuration. The handle 240 preferably is pivotally connected to the device 210. Preferably, one or more handle positions (shown by the phantom line handle positions in FIG. 6) may be provided for convenience of use. The device 210 may include a fastening mechanism 217 with a pivot connection 218. The fastening mechanism 217 preferably is configured to permit the handle 240 to be secured in one or more positions.

Preferably, the heated air output unit 200 may be provided with a control mechanism for facilitating the regulation of the heat intensity, the velocity of the air, or both. For example, according to a preferred embodiment, a control mechanism is shown having a first regulator 241 for controlling the operation of the air velocity speed, such as, for example, off, low, medium and high position selections, which may be indicated with indicia 242. A second regulator 243 is shown for regulating the heat of the air. Corresponding indicia 244 may be provided to indicate the selections of the second regulator 243, such as the indicia indicating low, medium and high heat selections. Preferably, the heat and velocity controls, such as the regulators 243, 244, are provided proximate to the handle 240 so that a user may hold the device 210, and at the same time, be able to control the air delivery, such as, the air speed and the air temperature.

Preferably, the heated air output unit 200 is configured to work in conjunction with a power source, such as conventional household current, or alternately, a battery, or rechargeable power supply. A power cord 201 and plug 202 may be provided, to include a safety plug with reset button in the event of an electrical current disturbance. A heating mechanism (not shown), such as, for example, ceramic heating components, heating elements, or other heating devices, may be employed to provide a source for heating air to be drawn through the unit 200, and then drawn through the delivery component 205. Preferably, a fan (not shown) is provided to force air through the delivery component 205 for delivery of heated air through the nozzle 211 of the device 210.

Though the control regulators are described and illustrated for controlling the heat intensity and the air velocity, according to alternate embodiments, a single regulator, with combined setting options may be provided (e.g., low heat, medium air flow speed). The heated air output unit 200 preferably has a housing 203 that is provided to secure components therein, such as, for example, a heating element and fan, as well as controls, transformers circuitry, or other elements. The heated air output unit 200 may be constructed in accordance with hair dryers, in particular, the handheld types, which deliver a regulatable flow of heated air. The housing 203 preferably has vents 204 so that air may be drawn through the heated air output unit 200, heated with the heating mechanism and delivered through the nozzle 211. Though vents 204 are illustrated on the bottom of the housing 203, they may be located on other areas of the device 210, such as, for example, the side (not shown), as long as they may be unobstructed when the device 210 is in use. According to preferred embodiments, the webbed or screened vent 204 may be hingably connected to the housing 203, or alternately may be a friction tongue-and-groove fitted lid behind which is a housing (not shown). Alternatively, the housing 203 may be constructed to accommodate specially configured HEPA filters intended to filter allergens from the air before the air enters the heated air output unit housing 203.

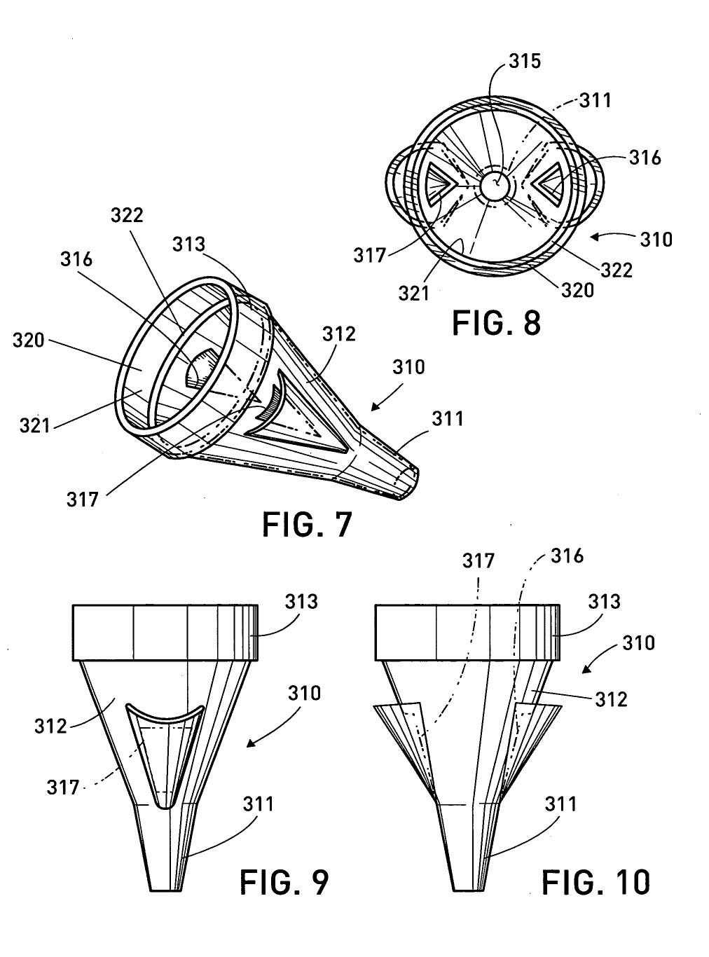

Referring to FIGS. 7-10, an alternate embodiment of a sinusitis treatment device 310 is shown. The sinusitis treatment device 310 has an upper body portion 312 and a lower body portion 313. The upper body portion 312 includes a wall 314. According to a preferred embodiment, the wall 314 forms the lower body portion 313. The device 310 includes a spout or nozzle 311 with an opening 315 through which heated air may pass. According to the preferred configuration illustrated in FIGS. 7-10, a ventilation mechanism is provided for venting a portion of the heated air flow from the device 310. The ventilation mechanism is illustrated in an exemplary configuration comprising apertures 316, 317 in the upper body portion 312, and may in addition have configured within the apertures 316, 317 an air flow regulating mechanism, or air pressure valve, designed to vent less air at lower air pressure speeds. As illustrated, the lower body portion 313 has a cylindrical configuration. A connection component 320 is provided and is illustrated including a circumferential wall portion 321 and a flange 322 formed on the lower body portion 313, which, for example, may be molded as a thicker portion of the circumferential wall portion 321. Preferably, the connection component 320 is configured to be releasably attached and detached from a nozzle of a commercially available hair dryer 100. The attachment may be accomplished through a friction fit between the circumferential wall portion 321 and the hair dryer nozzle. The connection component 320 preferably is made from materials, such as, for example, plastic, and more preferably, a heat resistant plastic, which may be positioned on the end of a hair dryer nozzle and held there while the hair dryer is used to deliver heated air through the device 310.

According to an alternate embodiment, the device 310 may be provided with a removable lower body portion 313, which may be attachable to and removable from the upper body portion 312. A plurality of lower body portions 313, each having a different diameter-size connecting component 320 for attachment to the diameter size of a given hair dryer nozzle 101, 101′, may be provided.

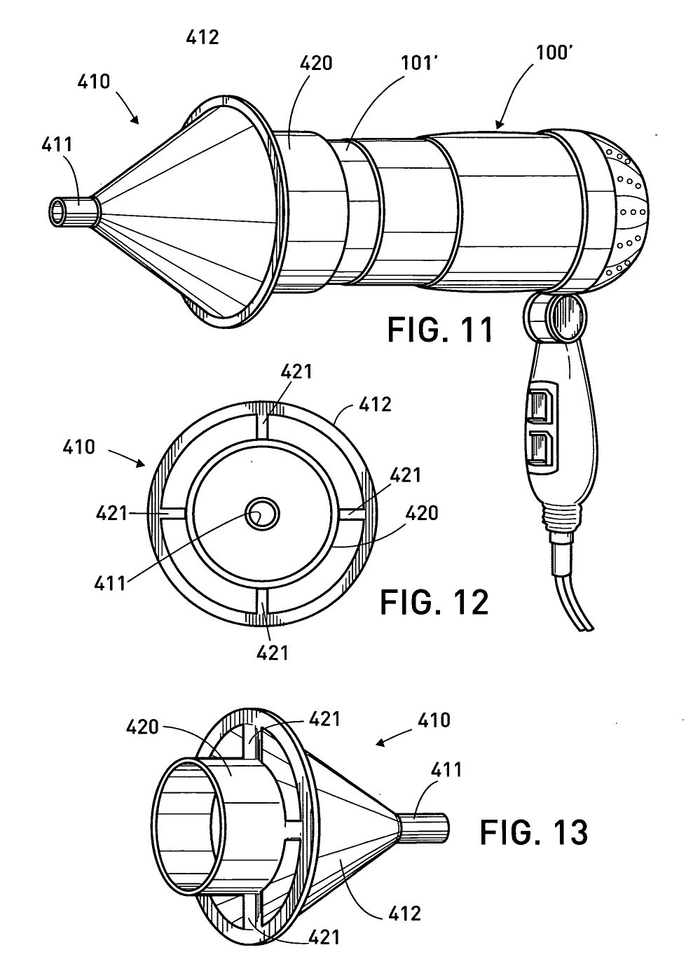

A fourth alternate embodiment of a sinusitis treatment device 410 is shown in FIGS. 11-13 having a body portion 412, a nozzle 411, a connecting flange 420 for connecting with a nozzle 101′ of a hair dryer 100′, and a plurality of connecting elements 421 that connect the connecting flange 420 with the body portion 412.

Preferably, the connecting flange 420 is dimensioned to fit on the nozzle of a hair dryer 101′. The connecting flange 420 may be configured having a tapered construction for accommodating nozzles, like that 101′, of different sizes and diameters.

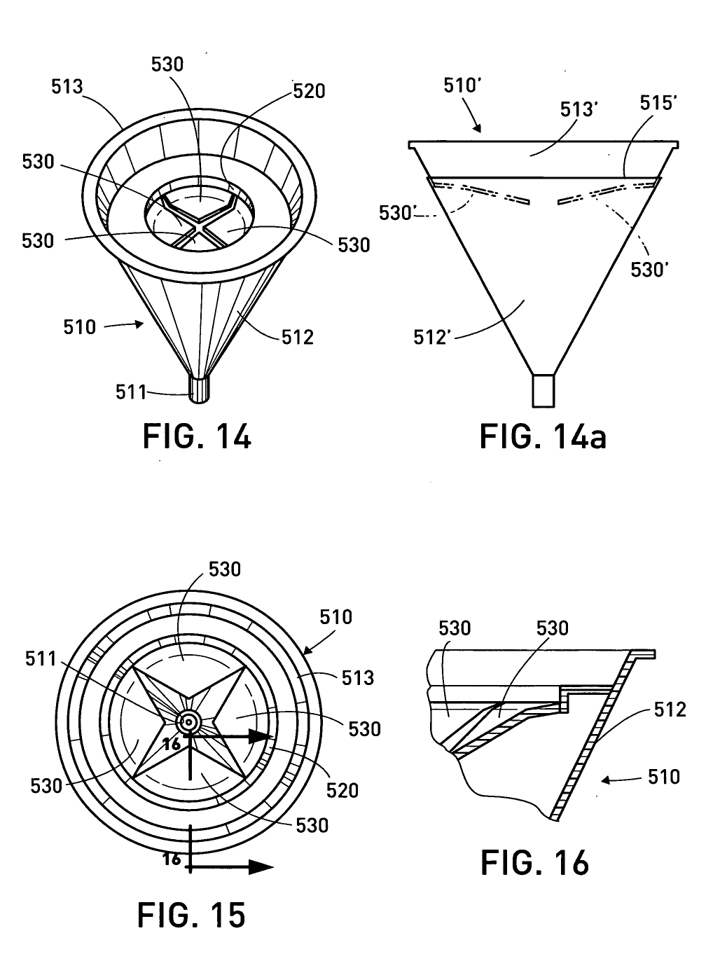

According to a fifth alternate embodiment illustrated in FIGS. 14-16, a sinusitis treatment device 510 is illustrated having a body portion 512, a nozzle 511, a connecting flange 520 for connecting with a nozzle of a hairdryer (such as those nozzles 101, 101′ shown and described herein or other suitable hair dryer), and a plurality of flow regulating sections 530 provided in the path of the nozzle to regulate air flow through the device 510 and through the nozzle 511. Sections 530, partially shown in isolation in FIG. 16, are configured as triangular flaps that preferably may be constructed from plastic or other suitable material and which possess a sufficient thickness and flexibility to function as a gripping device to hold the delivery device 510 firmly in place on the nozzle 101, 101′ of a hair dryer when the nozzle 101, 101′ is inserted through the flaps 530. FIGS. 14-16 illustrate a preferred arrangement where four flaps 530 are provided.

According to an alternate embodiment, as shown in FIG. 14a, four triangular plastic flaps 530′ that are intended for holding the device 510′ onto a variety of size hair dryer nozzles 101, 101′ are provided on a removable cylindrical cap 513′ that is configured to be held on the body portion 512 so that the cap 513′ may be readily removed, or cleaned and replaced. The removable cylindrical cap 513′ may be manually friction-fitted onto the cylindrical lip 515′ of the body portion 512′ of the device 510′ whenever desired for implementation. The cap 513′ preferably has flaps 530′ which may be configured similar to those flaps 530 of the device 510 shown in FIGS. 14, 15 and 16. According to alternate embodiments, the removable cap may be dimensioned so that a plurality of caps may be supplied where each cap is configured to fit onto a particular nozzle size or shape. A kit having a body portion 512′ and one or more caps 513′ may be provided. The removable connecting portion, or cap 513′, may be constructed to fit on the body portion 512′, preferably at the lower opening where the heated air enters the device 510′. The design of the removable connecting cap 513′ is not limited to that shown on FIG. 14a, and, according to an alternate embodiment, for example, may be configured to connect substantially flatly over cylindrical lip 515′ much the way a plastic lid commonly fits upon a cardboard or polystyrene foam coffee cup. These and alternate attachment mechanisms for the removable connecting cap 513′ may be utilized in connection with the sinusitis treatment devices shown and described herein, including, in particular, the exemplary embodiment illustrated in FIG. 14a.

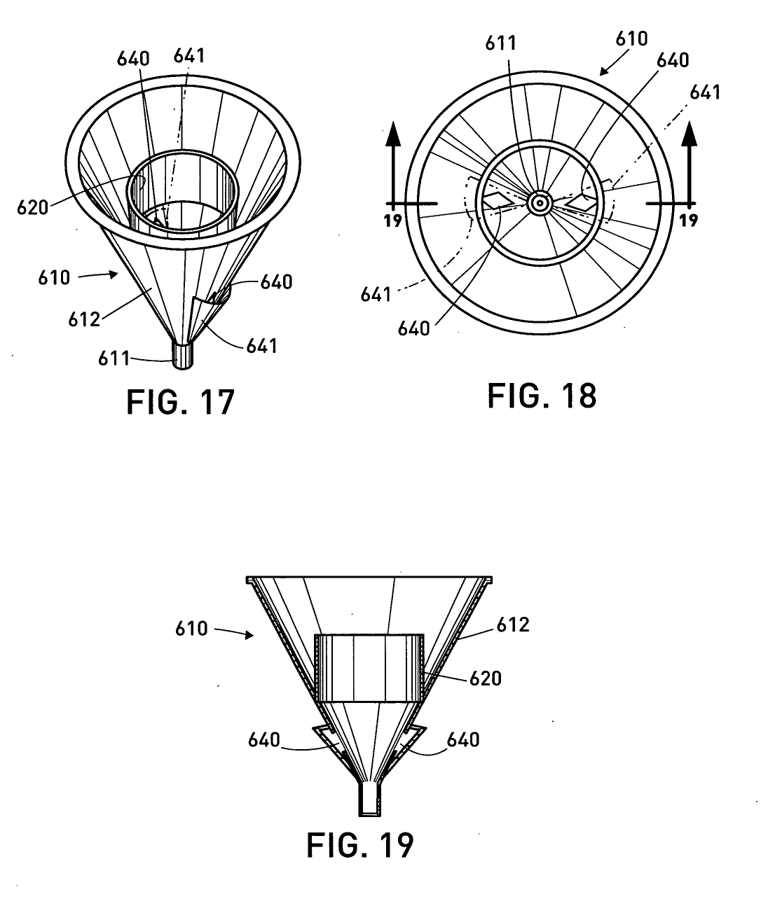

According to a sixth alternate embodiment illustrated in FIGS. 17-19, a sinusitis treatment device 610 is illustrated having a body portion 612, a nozzle 611, a connecting flange 620 for connecting with a nozzle of a hairdryer (such as those nozzles 101, 101′ shown and described herein or other suitable hair dryer) whereby the nozzle 611 slides snugly into sleeve-like flange 620 which is molded to the body portion 612 and offering no air venting normally offered by the funnel-like flared design of the device 610. Vent apertures 640 may therefore be provided, allowing excess air to escape the device 610 to avoid the overheating and damaging of the hair dryer.

Though not shown, according to preferred embodiments, a stop may be provided in connection with the embodiments of the devices in FIG. 1-6, so that the depth of insertion of the hair dryer nozzle within the device is controlled. This may be done to prevent the hair dryer nozzle from engaging the interior of the wall 13, 113 of the device 10, 110, respectively, so that ventilation of air is not obstructed. The stop may comprise flange sections disposed circumferentially at a location on the interior of the wall 13, 113, or may, for example, comprise a cross-piece that may restrict further penetration of the nozzle. An example of flange sections circumferentially located in this manner is illustrated by the alternate embodiment shown in FIGS. 14-16, where the device 510, may be configured to utilize the regulating sections 530 to not only affix the device 510 to a hair dryer nozzle 101, 101′, but also to prevent the nozzle from intruding so far into the body of 512 as to engage with the inner wall of the body portion 512, and thereby prevent the ventilation necessary to protect the hair dryer.

The sinusitis treatment devices shown and described herein may be molded as an integral component, or may be constructed from one or more elements. Preferably, a material is used that resists heat.

The following are examples of treatment applied to an individual to facilitate alleviation of a sinusitis condition that the individual, prior to the treatment, had been known to have.

### EXAMPLE 1

While breathing through the mouth, a warm flow of air from a hair dryer was directed up into the nostrils, tilting the head alternately to direct the flow more concentratedly into one nostril for a few minutes, and then for a few minutes into the other nostril, adjusting the heat level and the air force velocity to tolerable levels of comfort. The challenge of enduring the air blowing on the face and having to avoid scorching the nostrils was rewarded by the individual experiencing immediate relief and abatement of sinusitis symptoms. The individual was able to sleep without coughing from the first night of this procedure.

### EXAMPLE 2

The treatment of example 1 was followed both in the morning and in the evening for one month. These treatments with the hair dryer were sufficient to make the individual feel asymptomatic, and able to function during the day like a person without symptoms of sinusitis. The treatments also enabled the individual to reliably sleep at night.

The heat, sometimes as much as 140 degrees, was comfortable enough for several minutes and seemed to have a soothing effect on aching sinuses, although collateral air blowing on the face had become a nuisance and the nose tip and nostril edges; were becoming slightly red.

### EXAMPLE 3

The treatment, as in Example 2 was undertaken using a delivery device for directing the heated air, a small plastic funnel obtained from a hardware store. The funnel spout (or narrow end) was inserted into each nostril at intervals, allowing the flare of the funnel to protect the individuals face from the hot air stream that may otherwise chafe the skin near the nose area. The funnel directed the hot air stream more deeply into the individual's sinus than in Examples 1 and 2. The individual was able to administer the treatment with one hand holding the funnel and the other holding the hair dryer. This enabled the individual to control the degree of heat and the force level of the air, thereby sustaining longer treatment sequences in each nostril. This procedure was performed for approximately twenty minutes each morning and evening, alternating nostrils at three to four minute intervals, and resulted in great relief of the sinusitis symptoms. The individual was able, for another month, to sleep without coughing at night, and could confidently attend public events during the day without manifesting disturbing symptoms.

### EXAMPLE 4

Example 3 procedures were carried out, but with the mechanical adaptation of a small strip of electrical tape wrapped around the external surface at the tip of the funnel's spout. The electrical tape protected the edges of the individual's nostrils and the inner skin of the nose from the heat of the funnel. With the funnel, the individual was able to regulate the delivery of the air stream, and consequently the heat, by moving the relative spacing and positioning of the hair dryer (hot air source) and the funnel. This procedure continued to result in the relief of the sinusitis symptoms, and the individual was able to sleep at night and be symptom free in public for the next three months.

### EXAMPLE 5

Example 5 includes an effort to find a way to attach the funnel to the hair dryer nozzle so that the individual would not need both hands to perform the treatment. This effort was immediately abandoned when, upon pressing the funnel's inner wall tightly against the fair dryer nozzle while operating the hair dryer, the machine overheated and would no longer operate. A new Revlon model was immediately obtained and used for treatment as in Example 4, with continuing positive results for the next nine months. Further attempts to mechanically unite the funnel to the hair dryer were suspended pending the ability to manufacture a funnel that can be attached to a hair dryer while allowing ventilation while in operation.

A preferred treatment regimen involves administering the treatment after the individual wakes up each morning and before the individual goes to bed at night. In the beginning, and depending on the severity of the sinusitis condition, discipline may be necessary to not skip the morning treatment. One can easily be deceived, having slept the whole night without symptoms and feeling fine upon rising due to the treatment the night before. One may regret skipping the morning treatment should the sinusitis symptoms reappear later in the day when the individual may not be able to administer another treatment.

Some individuals may skip an evening treatment. However, if an evening treatment is skipped, it may result, for some individuals, in the return of sinusitis symptoms. Over time, however, as the treatment results in better sinus health, one learns that he or she can safely diminish the frequency and duration of the treatments.

The actual procedure of the time spent and the temperatures used in the treatment have been quite variable. Generally, according to one preferred treatment method, the treatment may be applied by alternating between nostrils (while breathing through the mouth) at periods of about 4 minutes each, with a pause for another 4 minutes after completing one two-nostril sequence. Preferably, one session of the treatment may be comprised of at least three sequences. This comes to about 32 minutes. Typically, in the morning, a first treatment may be administered after the individual awakens, (e.g., while drinking morning coffee), and a second treatment following that (e.g., perhaps after a shower), and the third treatment following that (e.g., after getting dressed). At night, a similar treatment sequence may be performed (e.g., while watching television), or even, at times, adding extra sessions.

Adjustments may be made to the force settings. For example, on the hair dryer machine used in Example 5, are between “low” and “high,” and temperature settings of “cool,” “warm,” and “hot”. These settings may be varied in each sequence according to how the hot air delivery feels, and how the individual feels (e.g., whether symptomatic or asymptomatic at the time). A “cool” setting would be rarely used, although it is actually a mildly warm setting that those beginning the treatment (or those who have stopped and are resuming treatment) might appreciate. A preferred setting is the “warm” setting at the “low” force setting, but other settings include beginning a treatment session at the “low” force setting but using the “hot” temperature because it quickly penetrates with the soothing heat, and, for some individuals, may instantly relieve any sinus pressure and pain. As the heat starts to get uncomfortable, the heat setting may simply be reduced in mid treatment (e.g., from “hot” to “warm”). However, according to a preferred embodiment, with the heat setting reduced or at a “warm” temperature, the individual may be able to increase the force level to “high,” which can be felt penetrating quite deeply into the sinus, even to the other side of one's face. A setting of “hot” at “high” (for most hair dryers) would normally not be indicated since it cannot usually be tolerated for long, unless the individual is congested from a cold. The treatment tolerance levels for air force and higher temperatures may vary in comfort tolerance levels from day to day for an individual. The individual may adjust the settings to different levels as preferred for that individual throughout a four minute treatment sequence.

As anyone who has sinusitis knows, nighttime, for some reason, is when the sinuses truly erupt with merciless relentlessness. The sinusitis sufferer becomes accustomed to identifying the hope of rest with the feeling of helplessness. After hours of coughing and clearing one's throat, things may settle down enough to doze off. But this does not last for long. One learns how to cough and clear one's throat while half asleep. This may then be followed by one learning from his or her dentist that this frustration in slumber has caused the destructive grinding of teeth at night.

The apparatus, system and treatment method of the present invention, when used in accordance with the Examples, has enabled an individual receiving the treatment to turn off that merciless mucus faucet at will and no longer grind teeth at night. After fourteen months of steady treatment, the individual experienced no negative side effects. The hot air administration did not result in any harmful drying out of the sinus nor were any nose bleeds experienced.

One thing that the individual observed is that the morning treatment, administered soon after waking up, seems to loosen up previously unnoticed congestion, beginning with a curious mild runny-nose phenomenon. Some kind of very thin fluid with less of the viscosity of mucus, more like tears, was observed to be secreted and flow after the first sequence, and, later on, there seemed to be enough hidden mucus breaking up within the sinus passages that the individual undertook some vigorous snorting and throat clearing between sequences, which seemed to loosen up and push out any overnight sinus clogging, leaving the individual symptom free all day. This was accomplished without any saline spray or irrigation. The same phenomenon of a non-mucus fluid was noticed at the beginning and end of the night treatment. On some nights, especially during a time period when the individual is experiencing an allergy season, the period between beginning and end of a treatment session may involve some prolonged vigorous snorting and throat clearing, and can involve some extra treatment sequences. The use of the device, system and treatment method described herein was productive and resulted in effective sleep for the individual. In some instances, at evenings, when treatment was administered, the individual had experienced a quiet presence of non-viscous fluid inside the nasal passages. The individual, who had been prone to suffering from colds, used the device, system and treatment method, and did not catch a cold in the spring, where prior years the individual was known to have colds at that time.

It has been further observed that an individual who is diligent in performing this treatment morning and evening for up to three or four months, some less and some more according to the person, can possibly look forward to weeks of being symptom free without resorting to the treatment, utilizing it intermittently according to the emergence of tell-tale signs such as congestion, more frequent need for throat clearing, aching within the sinuses, or the first signs of coming down with a cold. The degree of sinus rehabilitation through use of this device, method and system may vary according to the person, but rehabilitation clearly does take place.

In accordance with the examples, the current hair dryer and plastic funnel, as well as the delivery component and heated air delivery units described and illustrated herein, represent a treatment method that is less cumbersome than the messy and tedious saline water irrigation methods of sinusitis treatment (which were generally unreliable and certainly ineffective in the long term of eliminating sinusitis symptoms).

According to a preferred embodiment, a device for facilitating the administration of heated air, according to a first embodiment, is sufficiently small and lightweight that it may be held in one hand so that the user's thumb may control the heat on a sliding switch, and small enough for casual portability. Another switch on the device may be provided for “off,” “low,” and “high” to regulate the air velocity. Other regulation switches may be provided on the device, consistent with the invention disclosed herein. It is also preferred that the device include an optional HEPA filtering system consistent with this disclosure.

According to a preferred embodiment, configurations of the device include a spout or nozzle that an individual may insert into each nostril consecutively, and which, according to a preferred exemplary configuration, allows for about three quarters of an inch penetration and a diameter at the end of the spout of at least 6/16^{th} of an inch to ensure sufficient snugness to prevent air intended for the sinus cavities from escaping around the edges. Though a preferred spout dimension may universally accommodate most individuals, according to alternate embodiments the spout configuration and dimensions may be smaller or larger to correspond with the physiology of a particular individual. Preferably, the spout is constructed from a material that does not conduct heat, or at least, the spout is coated with a non heat conductive (or minimally heat conductive) material so that the tip of the spout may protect the nostril skin tissue. Examples of the covering for the nozzles or spouts include plastic tape (e.g., electrical tape), cloth tape, and the like. Molded nozzle caps (not shown) also may be utilized.

Accordingly, a measure of maintaining the hygiene of the device should be applied, such as washing the device, swabbing it with alcohol, or other cleaning applications. According to preferred embodiments, the spout may be provided as a removable component of the device (removable from the hot air source) and may be washed, or even placed in a dishwasher. A plurality of nozzles or spouts, and in different colors, may be used or provided in a kit with the hot air source so that a user may clean one or more spouts while using clean ones for an application, or select an appropriate sized or colored spout for use based on user compatibility. Though not shown, the spouts or nozzles may be separately attachable and detachable from the delivery component body portion, such as, for example, with the use of threads, and optionally gaskets, so that a plurality of nozzle or spout sizes and colors may be provided and selected for use, depending on the physiology and/or identification preferences of the individual.

While the invention has been described with reference to specific embodiments, the description is illustrative and is not to be construed as limiting the scope of the invention. Although embodiments of the device are illustrated having a single nozzle, according to alternate embodiments, the device may be constructed with two nozzles, for placement of the device into both nostrils of an individual. For example, the dual nozzle embodiments may be used in conjunction with the single nozzle embodiments, including where in a course of treatment, during a subsequent treatment, the two nozzle embodiment is used after the individual has used the single nozzle embodiment on one or both nostrils. Various modifications and changes may occur to those skilled in the art without departing from the spirit and scope of the invention described herein and as defined by the appended claims.

What is claimed is:

1. A system for facilitating treatment of a sinusitis condition comprising:
a) a heat source configured to deliver an output of heated air;
b) a delivery component having a space through which the heated air output of the heat source may pass;
c) wherein the delivery component is configured for comfortable placement in a nostril of an individual.

2. The system of claim 1, wherein the delivery component includes a handle.

3. The system of claim 1, wherein said delivery component includes an end that when exposed to heat, remains at a temperature that will not cause a burn of human skin.

4. The system of claim 3, wherein said delivery component end is plastic.

5. The device of claim 1, including flow velocity regulating means for regulating the velocity of the flow of heated air.

6. The system of claim 1, including heat regulating means for regulating the temperature of the heated air.

7. The system of claim 5, including heat regulating means for regulating the temperature of the heated air.

8. The system of claim 1, wherein the heat source comprises a hair dryer adapted to carry a removable delivery component thereon, and wherein said delivery component is configured with connecting means for connecting with said hair dryer.

9. The system of claim 1, wherein the delivery component includes a baffle that is regulatable to regulate air flow through the delivery component.

10. A device for facilitating treatment of a sinusitis condition comprising:
a) a delivery component having a space through which the heated air output of a heat source may pass;
b) wherein the delivery component is configured for placement in a nostril of an individual.

11. The device of claim 10, wherein said delivery component includes an end that when exposed to heat, remains at a temperature that will not cause a burn of human skin.

12. The device of claim 3, wherein said delivery component end is plastic.

13. The device of claim 10, wherein said delivery component is configured with connecting means for connecting with a standard household hair dryer.

14. The device of claim 10, wherein the delivery component includes at least one vent provided therein.

15. The device of claim 10, further including a heat source configured to deliver an output of heated air through said delivery component.

16. The device of claim 10, wherein the delivery component is detachable from and attachable to said heat source.

17. The device of claim 16, wherein said delivery component includes a connector for connecting said delivery component to a heat source.

18. The device of claim 17, wherein said connector comprises an elastomeric band.

19. The device of claim 18, wherein said delivery component has a wall and wherein said connector includes securing means for securing said elastomeric band to said wall.

20. The device of claim 10, including flow velocity regulating means for regulating the velocity of the flow of heated air.

21. The device of claim 10, including heat regulating means for regulating the temperature of the heated air.

22. The device of claim 20, including heat regulating means for regulating the temperature of the heated air.

23. The device of claim 10, wherein said delivery component is further configured to include a widened body, or flaired portion that shields the user's face from exposure to direct air delivery.

24. A method for administering a treatment to facilitate treating or preventing a sinusitis condition, the method comprising: the delivery of a flow of heated air through one nostril at a time into the individual's sinuses.

25. The method of claim 24, wherein the method includes:
a) providing the system of claim 1;
b) placing the delivery component on the individual's body in a position to deliver the flow of heated air to the individual's sinuses through one nostril at a time of the individual to whom the treatment is to be administered;
c) operating the heat source to deliver a flow of heated air through the delivery component and into one of the individual's nostrils at a time.

26. The method of claim 25, wherein the delivery component includes one nozzle, and the method includes placing the nozzle in a nostril of the individual.

27. The method of claim 25, wherein the delivery of heated air to the individual's sinuses through one nostril at a time is repeated over one or more sessions.

28. The device of claim 10, wherein the delivery component includes means of air pressure venting.

29. The device of claim 1, wherein said heat source output component includes a HEPA filter system.

30. The device of claim 14, including an elastomeric component for connecting to a nozzle of a conventional hair dryer.

31. The method of claim 24, wherein the treatment is administered in a plurality of sessions during a day.

32. The device of claim 10, wherein the device includes a plurality of movable flaps for connecting the device to a nozzle of a conventional hair dryer.

33. The device of claim 13, wherein the delivery component has a body portion and a removable connecting portion for connecting to a heated air delivery source.

34. The device of claim 33, wherein flaps are provided on said removable connecting portion that is configured to be attached to and detached from the body portion.

35. The device of claim 14, wherein said vent is covered to deflect air away from the user.

36. The device of claim 35, wherein the vent is a regulatable vent.
